SEQUENCE LISTING (24 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - DNA, 15 bp, Homo sapiens
tggcagtgctctgtt

SEQ ID NO: 22 - DNA, 15 bp, Homo sapiens
tggcagtactctgtt

SEQ ID NO: 23 - DNA, 18 bp, Homo sapiens
ggaccgagggagccaatg

SEQ ID NO: 24 - DNA, 18 bp, Homo sapiens
ggacgagggagccaatgc

(19) United States
(12) Patent Application Publication
(10) Pub. No.: US 2018/0237509 A1
(43) Pub. Date: Aug. 23, 2018

(54) METHODS OF TREATING ADAMTS13 DEFICIENCIES AND CONGENITAL THROMBOTIC THROMBOCYTOPENIA IN PEDIATRIC PATIENTS

(71) Applicant: Alexion Pharmaceuticals, Inc., New Haven, CT (US)

(72) Inventors: Marina NORIS, Nembro (IT); Giuseppe REMUZZI, Bergamo (IT); Carmine PECORARO, Naples (IT)

(73) Assignees: Alexion Pharmaceuticals, Inc., New Haven, CT (US); Alexion Pharmaceuticals, Inc., New Haven, CT (US)

(21) Appl. No.: 15/762,207

(22) PCT Filed: Sep. 23, 2016
(86) PCT No.: PCT/IB2016/001419

§ 371 Date: Mar. 22, 2018

Related U.S. Application Data
(60) Provisional application No. 62/235,618, filed on Oct. 1, 2015.
(60) Provisional application No. 62/233,630, filed on Sep. 28, 2015.

(51) Int. Cl. C07K 16/18; A61K 45/06; A61K 39/395; A61K 39/095

(52) U.S. Cl. CPC C07K 16/18; A61K 45/06; A61K 39/3955; A61K 2039/545; C07K 2317/565; C07K 2317/56; A61K 39/095

(57) ABSTRACT

Provided are methods for clinical treatment of an ADAMTS 13 deficiency by administering an anti-C5 antibody, or antigen binding fragment thereof. Also, provided are methods for clinical treatment of congenital Thrombotic Thrombocytopenic Purpura by administering an anti-C5 antibody, or antigen binding fragment thereof.

### RELATED APPLICATIONS

This application claims priority to and the benefit of U.S. Provisional Patent Application No. 62/233,630, filed on Sep. 28, 2015, and U.S. Provisional Patent Application No. 62/235,618, filed on Oct. 1, 2015, the disclosures of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Sep. 15, 2016, is named AXJ-203PC_SL.txt and is 33,699 bytes in size.

### BACKGROUND

Thrombotic thrombocytopenic purpura (TTP) is a rare disease that features thrombocytopenia, microangiopathic hemolytic anemia, and widespread microvascular thrombi that result in multiorgan dysfunction (see Galbusera M, et al., Semin Thromb Hemost. 2006; 32(2):81-89). Neurologic injury is common and has historically been used to differentiate TTP from atypical hemolytic uremic syndrome (aHUS), a related thrombotic microangiopathy in which acute kidney injury is a prominent feature (see Noris M, Remuzzi G. et al., N. Engl. J. Med. 2009; 361(17):1676-1687). However, acute and chronic kidney disease may be seen in patients with TTP and aHUS can involve extrarenal manifestations, so it can be difficult to discern the two diseases solely by clinical presentation (see Cataland S R, Wu H M, Blood Rev., 2014; 28(2):67-74). TTP is associated with a deficiency in ADAMTS13, a plasma metalloprotease that cleaves von Willebrand factor (vWF) multimers, with the consequent appearance of ultralarge vWF (ULvWF) multimers in the blood circulation (see Tsai H M, Int. J. Hematol. 2010; 91(1):1-19). ADAMTS13 deficiency in TTP is generally due to autoantibodies that typically are no longer detectable during remission. In 5% to 10% of cases, the enzymatic deficiency is congenital and caused by mutations in the ADAMTS13 gene (see George J N, Blood. 2010; 116(20):4060-4069). The mainstay of therapy in congenital TTP is fresh frozen plasma infusions or plasma exchange to supply enough ADAMTS13 protein to cleave the ULvWF multimers (see George J N, Blood. 2010; 116(20):4060-4069). In patients with recurrent congenital TTP, prophylactic fresh frozen plasma is often administered every 2 to 3 weeks to maintain ADAMTS13 levels high enough to cleave the ULvWF multimers and prevent the formation of microthrombi (see George J N, Blood. 2010; 116(20):4060-4069). However, plasma treatment is associated with morbidity and mortality, including the acute risk for allergic reactions/anaphylaxis and transfusion-related acute lung injury and the long-term risk for infection (historically with hepatitis B virus, hepatitis C virus, and HIV and more recently with the prion-associated Creutzfeldt-Jacob disease, which is resistant to current inactivation procedures (see Scully M., Transfus Apher. Sci. 2014; 51(1):11-14). Accordingly, it is an object of the present invention to provide improved methods for treating patients (in particular, pediatric patients) with an ADAMSTS13 deficiency and/or congenital TTP.

### SUMMARY

Provided herein are compositions and methods for treating an ADAMSTS13 deficiency in a human pediatric patient, comprising administering to the patient an anti-C5 antibody, or antigen binding fragment thereof. Also provided are compositions and methods for treating congenital TTP in a human pediatric patient, comprising administering to the patient an anti-C5 antibody, or antigen binding fragment thereof. In one embodiment, the anti-C5 antibody, or antigen binding fragment thereof, is administered (or is for administration) according to a particular clinical dosage regimen (i.e., at a particular dose amount and according to a specific dosing schedule).

An exemplary anti-C5 antibody is eculizumab comprising heavy and light chains having the sequences shown in SEQ ID NOs: 10 and 11, respectively, or antigen binding fragments and variants thereof. In other embodiments, the antibody comprises the heavy and light chain CDRs or variable regions of eculizumab. In another embodiment, the antibody comprises the CDR1, CDR2, and CDR3 domains of the VH region of eculizumab having the sequence set forth in SEQ ID NO: 7, and the CDR1, CDR2 and CDR3 domains of the VL region of eculizumab having the sequence set forth in SEQ ID NO: 8. In another embodiment, the antibody comprises heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. In another embodiment, the antibody comprises VH and VL regions having the amino acid sequences set forth in SEQ ID NO: 7 and SEQ ID NO: 8, respectively.

Another exemplary anti-C5 antibody is antibody BNJ441 (also known as ALXN1210) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of antibody BNJ441. In another embodiment, the antibody comprises the CDR1, CDR2, and CDR3 domains of the heavy chain variable (VH) region of antibody BNJ441 having the sequence shown in SEQ ID NO:12, and the CDR1, CDR2 and CDR3 domains of the light chain variable (VL) region of antibody BNJ441 having the sequence shown in SEQ ID NO:8. In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively.

In another embodiment, the antibody comprises VH and VL regions having the amino acid sequences set forth in SEQ ID NO:12 and SEQ ID NO:8, respectively.

In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

Another exemplary anti-C5 antibody is antibody BNJ421 comprising heavy and light chains having the sequences shown in SEQ ID NOs:20 and 11, respectively, or antigen binding fragments and variants thereof. In another embodiment, the antibody comprises the heavy and light chain CDRs or variable regions of BNJ421. In another embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

In another embodiment, the antibody competes for binding with, and/or binds to the same epitope on C5 as, the above-mentioned antibodies. In another embodiment, the antibody has at least about 90% variable region amino acid sequence identity with the above-mentioned antibodies (e.g., at least about 90%, 95% or 99% variable region identity with SEQ ID NO:12 and SEQ ID NO:8).

Accordingly, in one aspect, methods of treating a human pediatric patient with an ADAMSTS13 deficiency and/or congenital TTP are provided, the methods comprising administering to the patient an effective amount of an anti-C5 antibody, or antigen binding fragment thereof. In one embodiment, the dose of the anti-C5 antibody, or antigen binding fragment thereof, is a flat-fixed dose that is fixed irrespective of the weight of the patient. For example, the anti-C5 antibody, or antigen binding fragment thereof, may be administered at a fixed dose of 900 mg or 1,200 mg, without regard to the patient's weight. In certain embodiments, dosage regimens are adjusted to provide the optimum desired response (e.g., an effective response).

In one embodiment, the anti-C5 antibody, or antigen binding fragment thereof, is administered (a) weekly at a dose of 900 mg for four weeks and (b) once every two weeks thereafter at a dose of 1,200 mg.

In one embodiment, the anti-C5 antibody, or antigen binding fragment thereof, is administered for at least 20, 21, 22, 23, 24, 25, 26, 27, 28, 29, 30, 31, 32, 33, 34, 35, 40, 45, 50, 55, or 60 weeks. In another embodiment, the anti-C5 antibody, or antigen binding fragment thereof, is administered for at least one, two, three, four, five, or six years.

The anti-C5 antibodies, or antigen binding fragments thereof, can be administered to a patient by any suitable means. In one embodiment, the antibodies are formulated for intravenous administration.

In addition, the patient can be administered one or more suitable therapeutic agents, prior to administration of the anti-C5 antibodies, or antigen binding fragments thereof. For example, in one embodiment, the patient is administered an antimeningococcal vaccine prior to treatment with the anti-C5 antibody, or antigen binding fragment thereof. In another embodiment, the patient is administered one or more antibiotics prior to treatment with the anti-C5 antibody, or antigen binding fragment thereof.

In one aspect, methods of treating a human pediatric patient with an ADAMSTS13 deficiency are provided, the methods comprising administering to the patient an effective amount of an anti-C5 antibody, or antigen binding fragment thereof. In one embodiment, the ADAMTS13 deficiency is associated with one or more ADAMTS13 gene mutations. For example, in one embodiment, the ADAMTS13 mutation is a guanine to adenine change at nucleotide 3,251 (which is predicted to cause a cysteine to tyrosine substitution at amino acid 1,084). In another embodiment, the ADAMTS13 mutation is a deletion of a cytosine at nucleotide 4,049 (resulting in a frameshift after the arginine at amino acid 1,351, which is predicted to lead to a premature stop codon 9 amino acids later. In another embodiment, the ADAMTS13 deficiency is associated with two ADAMTS13 mutations, wherein the first ADAMTS13 mutation is a guanine to adenine change at nucleotide 3,251 and the second ADAMTS13 mutation is a deletion of a cytosine at nucleotide 4,049. In another embodiment, the ADAMTS13 deficiency is determined by undetectable levels of ADAMTS13 activity, as assessed by a collagen-binding assay and/or fluorescence resonance energy transfer (FRET) (e.g., using the ADAMTS13 fluorogenic substrate FRETS-rVWF73).

In a particular embodiment, methods of treating a human pediatric patient with an ADAMTS13 deficiency, the method comprising intravenously administering eculizumab to the patient (a) weekly at a dose of 900 mg for four weeks and (b) once every two weeks thereafter at a dose of 1,200 mg, are provided.

In another particular embodiment, methods of treating a human pediatric patient with congenital TTP, the method comprising intravenously administering eculizumab to the patient (a) weekly at a dose of 900 mg for four weeks and (b) once every two weeks thereafter at a dose of 1,200 mg, are provided.

Prior to treatment, with the anti-C5 antibodies, or antigen binding fragments thereof, the patient may exhibit one or more particular characteristics. For example, in one embodiment, the human pediatric patient has ultra large von Wildebrand factor (ULvWF) multimers circulating in the blood prior to treatment. In another embodiment, the human pediatric patient has elevated levels of C3a in the plasma prior to treatment. In another embodiment, the human pediatric patient has elevated levels of sC5b-9 in the plasma prior to treatment. In another embodiment, the human pediatric patient has elevated levels of serum induced C5b-9 deposits on microvascular endothelial cells ex-vivo and C3 glomerular deposits in kidney biopsy specimens prior to treatment. In a further embodiment, the human pediatric patient has elevated levels of C5b-9 glomerular deposits in kidney biopsy specimens prior to treatment.

The efficacy of the treatment methods provided herein can be assessed using any suitable means. Patients treated according to the methods disclosed herein preferably experience improvement in at least one sign of an ADAMTS13 deficiency and/or congenital TTP. For example, the treatment may produce at least one therapeutic effect selected from the group consisting of increased then normalized platelet count, normalized lactate dehydrogenase (LDH) levels, normalized serum creatinine levels, and normalized diuresis. For example, in one embodiment, the treatment results in a normalized platelet count, normalized lactate dehydrogenase (LDH) and normalized dieresis within 3 days. In another embodiment, the treatment results in a normalized serum creatinine within 2 weeks. In another embodiment, lactate dehydrogenase (LDH) levels can be used to evaluate responsiveness to a therapy. In other embodiments, patients treated according to the disclosed methods experience reductions in LDH levels by about 20%, 30%, 40%, 50%, 60%, 70%, 80% or more compared to no treatment. In another embodiment, patients treated according to the disclosed methods experience reductions in LDH levels to near normal levels or to within 10%, or within 20% above what is considered the normal level.

Further provided are kits that include a pharmaceutical composition containing an anti-C5 antibody, or antigen binding fragment thereof, such as eculizumab, BNJ441, or BNJ421, and a pharmaceutically-acceptable carrier, in a therapeutically effective amount adapted for use in the methods described herein. In one embodiment, the kit comprises:

(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and

(b) instructions for using the anti-C5 antibody, or antigen binding fragment thereof, according to any of the methods described herein.

In another embodiment, the kit comprises:

(a) a dose of an anti-C5 antibody, or antigen binding fragment thereof, comprising CDR1, CDR2 and CDR3 domains of the heavy chain variable region having the sequence set forth in SEQ ID NO:7, and CDR1, CDR2 and CDR3 domains of the light chain variable region having the sequence set forth in SEQ ID NO:8; and

(b) instructions for using the anti-C5 antibody, or antigen binding fragment thereof, according to any of the methods described herein.

### BRIEF DESCRIPTION OF THE DRAWINGS

FIG. 1 is a timeline depicting early clinical events and initiation of therapy. “URT” refers to upper respiratory tract infection and “AKI” refers to acute kidney injury.

FIGS. 2A-2B are graphs depicting the Effect of eculizumab (Ecu) on platelet count (FIG. 2A) and serum-induced endothelial C5b-9 deposits in an ex vivo assay (FIG. 2B). Treatments shown in FIG. 2A include hemodialysis (HD) and Ecu doses (arrows). Results shown in FIG. 2B pertain to an ex vivo assay of serum-induced C5b-9 deposition on activated human microvascular endothelial cells.

FIG. 3 shows the rapid increase of platelet counts and restoration of diuresis in the patient following administration of the first dose of eculizumab (900 mg)

FIG. 4 depicts the recovery of kidney function after first eculizumab dose.

FIG. 5 depicts serum creatinine levels from day 0 to day 140.

FIG. 6 is a representation and localization of the two heterozygous ADAMTS13 mutations found in the patient. Figure discloses SEQ ID NOS 21, 23, 22, 24, 21, 23, 21, and 23, respectively, in order of appearance.

### DETAILED DESCRIPTION

### I. Definitions

As used herein, the term “subject” or “patient” is a human patient (e.g., a patient having an ADAMSTS13 deficiency and/or congenital TTP).

As used herein, the term “pediatric patient” refers to an infant, child, or adolescent from birth up to the age of 18.

As used herein, the term “congenital” refers to a condition associated with genetic defect present at birth (e.g., whether inherited or caused by the environment).

As used herein, “Thrombotic Thrombocytopenic Purpura” (also known as TTP or Moschcowitz syndrome) is a rare disorder of the blood-coagulation system, which causes extensive microscopic clots to form in the small blood vessels throughout the body (see, e.g., Moake J L (2002), N. Engl. J. Med. 347 (8): 589-600). These small blood clots, called thrombi, can damage many organs including the kidneys, heart and brain. Most cases of TTP arise from severely reduced activity of the enzyme ADAMTS13.

ADAMTS13 is a metalloprotease responsible for cleaving large multimers of von Willebrand factor (ULvWF) into smaller units. An ADAMTS13 deficiency results in circulating ULvWF multimers, which increase platelet adhesion to areas of endothelial injury, particularly at arteriole-capillary junctions (see Tsai H M, Int. J. Hematol. 2010; 91(1):1-19). ADAMTS13 deficiency in TTP is generally due to autoantibodies that typically are no longer detectable during remission. In 5% to 10% of cases, the enzymatic deficiency is congenital and caused by mutations in the ADAMTS13 gene (see George J N, Blood. 2010; 116(20):4060-4069).

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. Effective treatment may refer to alleviation of at least one symptom of an ADAMSTS13 deficiency and/or congenital TTP (e.g., peripheral thrombocytopenia, microangiopathic hemolytic anemia (MAHA) and/or single or multiple organ failure of variable severity).

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In one example, an “effective amount” is the amount of anti-C5 antibody, or antigen binding fragment thereof, clinically proven to alleviate at least one symptom of an ADAMSTS13 deficiency and/or congenital TTP. An effective amount can be administered in one or more administrations.

As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., the anti-C5 antibody, or antigen binding fragment thereof,).

The term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

### II. Anti-C5 Antibodies

The anti-C5 antibodies described herein bind to complement component C5 (e.g., human C5) and inhibit the cleavage of C5 into fragments C5a and C5b. Anti-C5 antibodies (or VH/VL domains derived therefrom) suitable for use in the invention can be generated using methods well known in the art. Alternatively, art recognized anti-C5 antibodies can be used. Antibodies that compete with any of these art-recognized antibodies for binding to C5 also can be used.

An exemplary anti-C5 antibody is eculizumab comprising heavy and light chains having the sequences shown in SEQ ID NOs: 10 and 11, respectively, or antigen binding fragments and variants thereof. Eculizumab (also known as Soliris®) is described in U.S. Pat. No. 6,355,245, the teachings or which are hereby incorporated by reference. Eculizumab is a humanized monoclonal antibody that is a terminal complement inhibitor.

In other embodiments, the antibody comprises the heavy and light chain CDRs or variable regions of eculizumab. Accordingly, in one embodiment, the antibody comprises the CDR1, CDR2, and CDR3 domains of the VH region of eculizumab having the sequence set forth in SEQ ID NO: 7, and the CDR1, CDR2 and CDR3 domains of the VL region of eculizumab having the sequence set forth in SEQ ID NO: 8. In another embodiment, the antibody comprises heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. In another embodiment, the antibody comprises VH and VL regions having the amino acid sequences set forth in SEQ ID NO: 7 and SEQ ID NO: 8, respectively.

Another exemplary anti-C5 antibody is antibody BNJ441 comprising heavy and light chains having the sequences shown in SEQ ID NOs:14 and 11, respectively, or antigen binding fragments and variants thereof. BNJ441 (also known as ALXN1210) is described in PCT/US2015/019225 and U.S. Pat. No. 9,079,949, the teachings or which are hereby incorporated by reference. BNJ441 is a humanized monoclonal antibody that is structurally related to eculizumab (Soliris®). BNJ441 selectively binds to human complement protein C5, inhibiting its cleavage to C5a and C5b during complement activation. This inhibition prevents the release of the proinflammatory mediator C5a and the formation of the cytolytic pore-forming membrane attack complex C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of BNJ441. Accordingly, in one embodiment, the antibody comprises the CDR1, CDR2, and CDR3 domains of the VH region of BNJ441 having the sequence set forth in SEQ ID NO:12, and the CDR1, CDR2 and CDR3 domains of the VL region of BNJ441 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in PCT/US2015/019225 and U.S. Pat. No. 9,079,949, the teachings or which are hereby incorporated by reference.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33(17/18):1389-1401] exemplifies the identification of CDR boundaries according to Kabat and Chothia definitions.

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), or enzyme-linked immunosorbent assay (ELISA). See, e.g., Benny K. C. Lo (2004) “Antibody Engineering: Methods and Protocols,” Humana Press (ISBN: 1588290921); Johne et al. (1993) J Immunol Meth 160:191-198; Jonsson et al. (1993) Ann Biol Clin 51:19-26; and Jonsson et al. (1991) Biotechniques 11:620-627.

In one embodiment, the antibody competes for binding with, and/or binds to the same epitope on C5 as, the antibodies described herein. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competing antibodies bind to the same epitope, an overlapping epitope or to adjacent epitopes (e.g., as evidenced by steric hindrance).

Anti-C5 antibodies, or antigen-binding fragments thereof described herein, used in the methods described herein can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246: 1275-1281 (1989).

### III. Compositions

Also, provided herein are compositions comprising an anti-C5 antibody, or antigen binding fragment thereof. In one embodiment, the composition comprises an antibody comprising the CDR1, CDR2, and CDR3 domains of the VH region of eculizumab having the sequence set forth in SEQ ID NO: 7, and the CDR1, CDR2 and CDR3 domains of the VL region of eculizumab having the sequence set forth in SEQ ID NO: 8. In another embodiment, the antibody comprises heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. In another embodiment, the antibody comprises VH and VL regions having the amino acid sequences set forth in SEQ ID NO: 7 and SEQ ID NO: 8, respectively.

In another embodiment, the antibody comprises the heavy and light chain CDRs or variable regions of BNJ441. In another embodiment, the antibody comprises the CDR1, CDR2, and CDR3 domains of the VH region of BNJ441 having the sequence set forth in SEQ ID NO:12, and the CDR1, CDR2 and CDR3 domains of the VL region of BNJ441 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively. In another embodiment, the antibody comprises

In another embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

The compositions can be formulated as a pharmaceutical solution, e.g., for administration to a subject for the treatment or prevention of a complement-associated disorder. The pharmaceutical compositions will generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt, sugars, carbohydrates, polyols and/or tonicity modifiers.

The compositions can be formulated according to standard methods. Pharmaceutical formulation is a well-established art, and is further described in, e.g., Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. For example, compositions containing a composition intended for systemic or local delivery can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

### IV. Outcomes

Provided herein are methods for treating an ADAMTS13 deficiency in a human pediatric patient comprising administering to the patient an anti-C5 antibody. Also provided are methods for treating congenital TTP in a human pediatric patient comprising administering to the patient an anti-C5 antibody. Symptoms of congenital TTP include, but are not limited to, profound peripheral thrombocytopenia (e.g., a decrease in platelets) microangiopathic hemolytic anemia (MAHA, e.g., a subgroup of hemolytic anemia (loss of red blood cells through destruction) caused by mechanical factors in the small blood vessels) and single or multiple organ failure of variable severity.

In one embodiment, the human pediatric patient has ultra large von Willebrand factor (ULvWF) multimers circulating in the blood prior to treatment. In another embodiment, the human pediatric patient has elevated levels of C3a in the plasma prior to treatment. In another embodiment, the human pediatric patient has elevated levels of sC5b-9 in the plasma prior to treatment. In another embodiment, the human pediatric patient has elevated levels of serum-induced C5b-9 deposits on microvascular endothelial cells ex vivo and C3 glomerular deposits in kidney biopsy specimens prior to treatment. In a further embodiment, the human pediatric patient has elevated levels of C5b-9 glomerular deposits in kidney biopsy specimens prior to treatment.

Patients treated according to the methods disclosed herein preferably experience improvement in at least one sign of an ADAMTS13 deficiency and/or congenital TTP. For example, the treatment may produce at least one therapeutic effect selected from the group consisting of a normalized platelet count, normalized lactate dehydrogenase (LDH) levels, normalized serum creatinine levels, and normalized dieresis. In one embodiment, the treatment results in a normalized platelet count, normalized lactate dehydrogenase (LDH) and normalized dieresis within 3 days. In another embodiment, the treatment results in a normalized serum creatinine within 2 weeks. In another embodiment, the platelet count increases and then normalizes. In another embodiment, lactate dehydrogenase (LDH) levels can be used to evaluate responsiveness to a therapy. In other embodiments, patients treated according to the disclosed methods experience reductions in LDH levels by about 20%, 30%, 40%, 50%, 60%, 70%, 80% or more compared to no treatment. In another embodiment, patients treated according to the disclosed methods experience reductions in LDH levels to near normal levels or to within 10%, or within 20% above what is considered the normal level.

### V. Kits and Unit Dosage Forms

Also provided herein are kits which include a pharmaceutical composition containing an anti-C5 antibody, or antigen binding fragment thereof, such as eculizumab, and a pharmaceutically-acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having an ADAMTS13 deficiency and/or congenital TTP. The kit also can include a syringe.

Optionally, the kits include multiple packages of the single-dose pharmaceutical compositions each containing an effective amount of the anti-C5 antibody, or antigen binding fragment thereof, for a single administration in accordance with the methods provided above. Instruments or devices necessary for administering the pharmaceutical composition(s) also may be included in the kits. For instance, a kit may provide one or more pre-filled syringes containing an amount of the anti-C5 antibody, or antigen binding fragment thereof.

In one embodiment, the present invention provides a kit for treating an ADAMTS13 deficiency in a human pediatric patient, the kit comprising:

(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and

(b) instructions for using the anti-C5 antibody, or antigen binding fragment thereof, according to any of the methods described herein.

In another embodiment, the present invention provides a kit for treating an ADAMTS13 deficiency in a human pediatric patient, the kit comprising:

(a) a dose of an anti-C5 antibody, or antigen binding fragment thereof, comprising CDR1, CDR2 and CDR3 domains of the heavy chain variable region having the sequence set forth in SEQ ID NO:7, and CDR1, CDR2 and CDR3 domains of the light chain variable region having the sequence set forth in SEQ ID NO:8; and

(b) instructions for using the anti-C5 antibody, or antigen binding fragment thereof, according to any of the methods described herein.

In another embodiment, the present invention provides a kit for treating congenital TTP in a human pediatric patient, the kit comprising:

(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and

(b) instructions for using the anti-C5 antibody, or antigen binding fragment thereof, according to any of the methods described herein.

In another embodiment, the present invention provides a kit for treating congenital TTP in a human pediatric patient, the kit comprising:

(a) a dose of an anti-C5 antibody, or antigen binding fragment thereof, comprising CDR1, CDR2 and CDR3 domains of the heavy chain variable region having the sequence set forth in SEQ ID NO:7, and CDR1, CDR2 and CDR3 domains of the light chain variable region having the sequence set forth in SEQ ID NO:8; and

(b) instructions for using the anti-C5 antibody, or antigen binding fragment thereof, according to any of the methods described herein.

The following examples are merely illustrative and should not be construed as limiting the scope of this disclosure in any way as many variations and equivalents will become apparent to those skilled in the art upon reading the present disclosure.

The contents of all references, Genbank entries, patents and published patent applications cited throughout this application are expressly incorporated herein by reference.

### EXAMPLES

### Example 1: Case Study of Pediatric Patient

Treatment and Observations

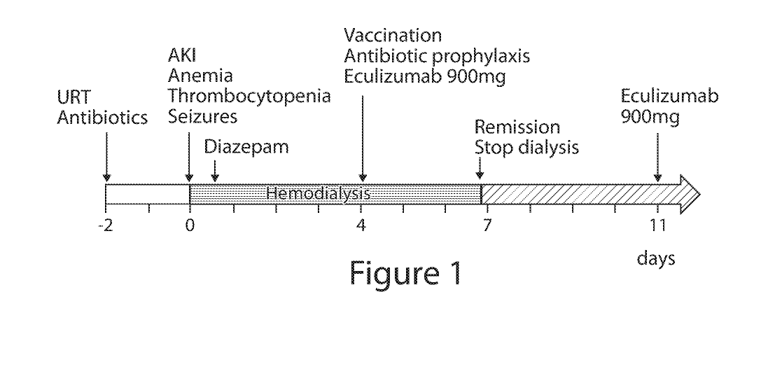

A 12-year-old Italian boy presented to the Pediatric Nephrology Unit of Santobono-Pausilipon Hospital in early 2012 with Coombs-negative hemolytic anemia (hemoglobin, 7.8 g/dL; lactate dehydrogenase, 1,449 IU/L; undetectable haptoglobin, and schistocytes in the blood smear), thrombocytopenia (platelet count, 9 3103/mL), acute kidney injury requiring hemodialysis (estimated glomerular filtration rate [eGFR], 7 mL/min/1.73 m2 as calculated using the bedside Schwartz equation (Schwartz G J, Work D F., Clin. J. Am. Soc. Nephrol. 2009; 4(11):1832-1843), and generalized seizures, preceded by an upper respiratory tract infection treated with antibiotics. Full laboratory values are provided in Table 1 and a timeline of the events immediately preceding and following admission is shown in FIG. 1

TABLE 1
Laboratory Parameters at Admission

| Parameter | Value | Reference Range |
|---|---|---|
| Sodium, mEq/L | 120 | 134-145 |
| Potassium, mEq/L | 5.8 | 3.5-5 |
| Calcium, mg/dL | 8.9 | 8.2-10.2 |
| Phosphorus, mg/dL | 8.2 | 2.5-4.5 |
| Creatinine, mg/dL | 9.31 | 0.5-1.2 |
| estimated glomerular | 7 | 80-120 |
| filtration rate (eGFR), | | |
| mL/min/1.73 m2 | | |
| Serum urea nitrogen, mg/dL | 364 | 10-20 |
| White blood cell count, | 8.3 | 4.5-11 |
| 3103/mL | | |
| Red blood cell count, | 3.6 | 4.5-6 |
| 3106/mL | | |
| Hemoglobin, g/dL | 7.8 | 14-18 |
| Platelets, 3103/mL | 9 | 150-400 |
| Lactate dehydrogenase, IU/L | 1,449 | 120-290 |
| Haptoglobin, g/L | 0.08 | 0.4-0.8 |

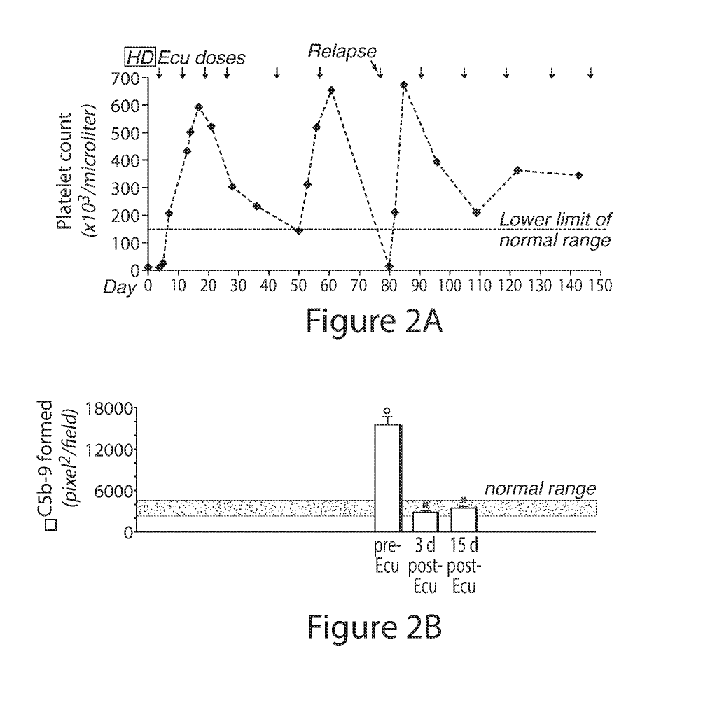

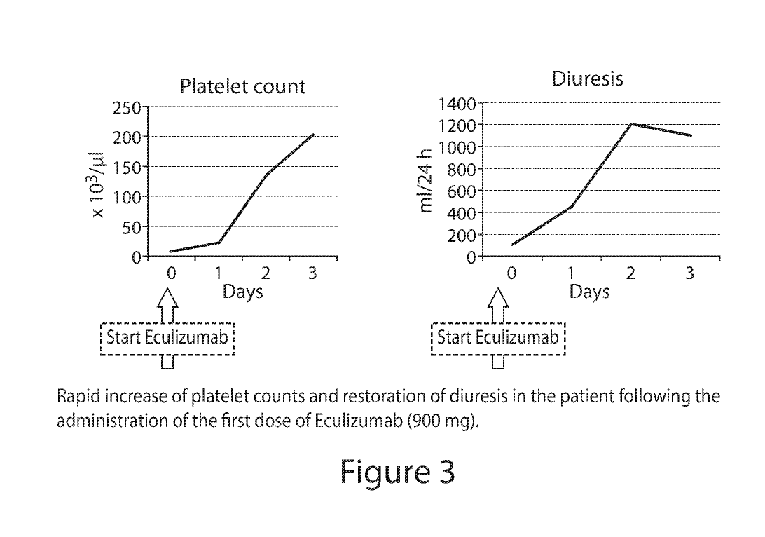

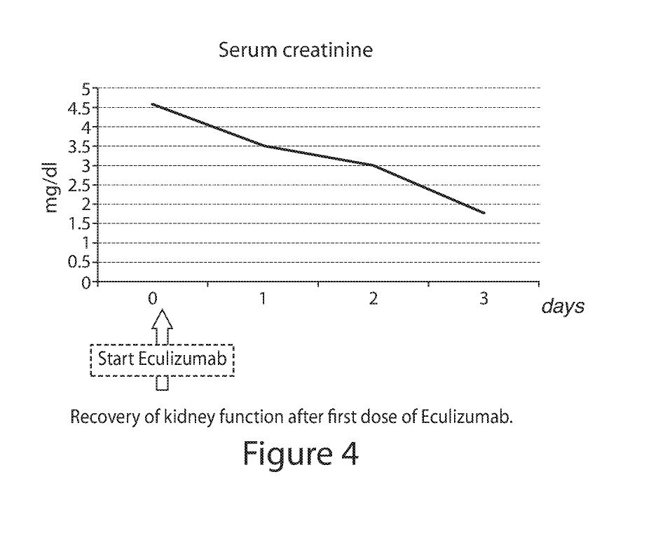

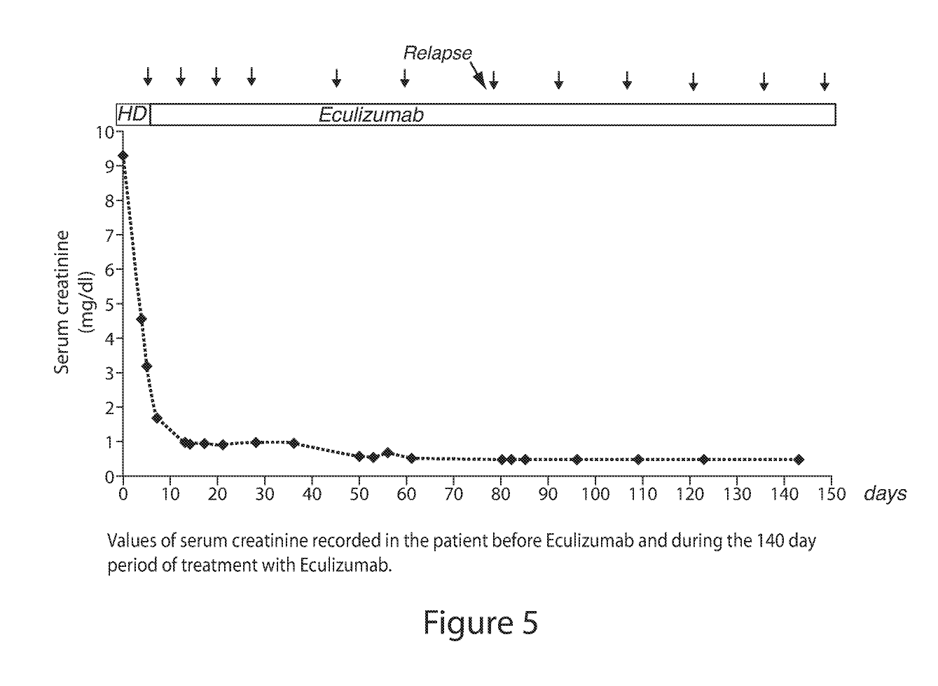

The childhood onset, severely decreased kidney function, absence of prodromal diarrhea, negative test results (from stool culture and serology) for Shiga-like toxin-producing Escherichia coli, and the elevated plasma level of the terminal complement complex sC5b-9 (520 ng/mL [reference range, <400 ng/mL], measured using the MicroVue SC5b-9 Enzyme Immunoassay [Quidel]) were all consistent with a diagnosis of complement-mediated aHUS (see Noris M, Remuzzi G., N. Engl. J. Med. 2009; 361(17):1676-1687). In order to rule out TTP, regulatory authority recommendations are to assay plasma ADAMTS13 activity before initiating eculuzumab treatment (see Agenzia Italiana del Farmaco. Pubblicazione schede di monitoraggio Registro SOLIRIS (SEUa). http://www.agenziafarmaco.gov.it/it/content/pubblicazione-schede-di-monito raggio-registro-soliris-seua-12012015). However, given the severity of the patient's clinical condition, the clinician started eculizumab (900 mg intravenous, 4 doses weekly; then 1,200 mg approximately every 2 weeks; FIG. 2) 4 days after admission and before the ADAMTS13 test results were available (antimeningococcal vaccination and antibiotic prophylaxis were administered before eculizumab initiation). The response was excellent (FIGS. 2-5) and within 3 days, the platelet count, lactate dehydrogenase level, and diuresis normalized. Accordingly, dialysis therapy was discontinued. Serum creatinine levels decreased to 1.78 mg/dL (eGFR, 36 mL/min/1.73 m2) and the severity of the patient's anemia lessened (hemoglobin, 8.9 g/dL). After the sixth eculizumab dose, an attempt to space out subsequent infusions resulted in thrombocytopenia (platelet count of 11 3103/mL, with diffuse petechial lesions) and microangiopathic hemolysis, with normal kidney function (eGFR, 115 mL/min/1.73 m2). At this time, ex vivo testing of the patient's serum showed elevated C5b-9 deposits on microvascular endothelial cells. Thus, eculizumab (1,200 mg) was reintroduced, promptly resolving the thrombocytopenia within 24 hours and the petechial lesions disappeared within 48 hours (FIG. 2).

After eculizumab treatment, ex vivo testing of the patient's serum no longer showed elevated C5b-9 deposits. Specifically, the results shown in FIG. 2B pertain to an ex vivo assay of serum-induced C5b-9 deposition on activated human microvascular endothelial cells. In brief, cells were incubated for 4 hours with serum (diluted 1:2 with test medium) from healthy controls or from the patient during the relapse before Ecu was administered (pre-Ecu) and 3 and 15 days after Ecu administration (post-Ecu). After incubation, cells were washed, fixed, and stained with an anti-human complement C5b-9 complex antibody followed by a fluorescently conjugated secondary antibody. Fluorescent staining on the endothelial cell surface (in pixel2/field) was calculated by analyzing 15 fields. The grey region shows the range of C5b-9 deposits induced by serum from healthy controls (n=3). ° P<0.001 versus control serum, *P<0.001 versus pre-Ecu. Data are mean±standard error.

The patient continued receiving eculizumab biweekly until day 140, at which point interdose intervals were lengthened until discontinuation. In the subsequent year, the patient had 5 hematologic relapses (without renal or neurologic symptoms), often associated with upper respiratory tract infections, each of which was effectively treated with a single dose of eculizumab. After a tonsillectomy in summer 2013, no further relapse occurred during a further 22-month drug-free follow-up.

While the patient was already receiving treatment, screening of aHUS-associated genes (CFH [complement factor H], CD46 [encoding membrane cofactor protein], CFI, CFB, C3, and THBD [encoding thrombomodulin]) was performed using next-generation sequencing on an Ion Torrent Personal Genome Machine (Life Technologies). This failed to show any mutation. However, the presence of genetic abnormalities in other complement-related genes could not be ruled out.

Both before and after eculizumab initiation, anti-CFH antibodies were undetectable by plasma enzyme-linked immunosorbent assay (performed as described by Dragon-Durey M A, et al., J. Am. Soc. Nephrol. 2005; 16(2):555-563). During the acute phase and also in remission, measurement of ADAMTS13 activity (in citrated plasma) showed undetectable levels (<6% using a collagen-binding assay and <3% by fluorescence resonance energy transfer [FRET] using the ADAMTS13 fluorogenic substrate FRETS-rVWF73), without evidence of inhibitory autoantibodies (see Palla R, et al., Thromb. Haemost. 2011; 105(2):381-385).

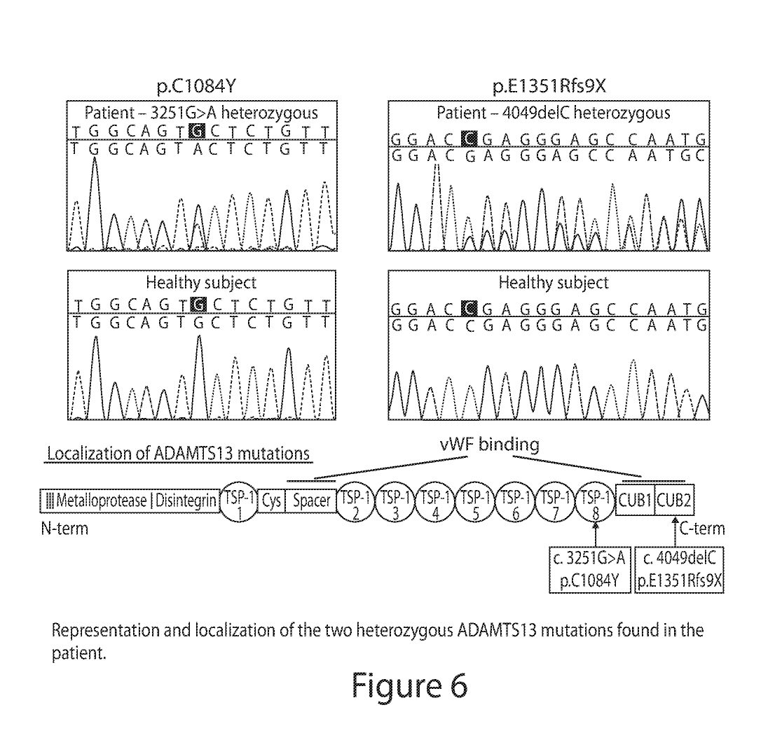

By sequencing ADAMTS13, 2 heterozygous mutations were detected. The first mutation is a guanine to adenine change at nucleotide 3,251 of the complementary DNA, which is predicted to cause a cysteine to tyrosine substitution at amino acid 1,084, and has been previously reported in patients with TTP (see, e.g., Loirat C, et al., Curr. Opin. Pediatr. 2013; 25(2):216-224; Lotta L A, et al., Blood. 2012; 120(2):440-448; and Hing Z A, et al., Br. J. Haematol. 2013; 160(6):825-837. The second mutation is a previously unpublished frameshift [from deletion of the cytosine at nucleotide 4,049 of the complementary DNA] after the arginine at amino acid 1,351, which is predicted to lead to a premature stop codon 9 amino acids later (see FIG. 6). Taken together, screening results were consistent with a diagnosis of congenital TTP.

In summary, this is the first case of congenital TTP treated with the complement inhibitor, eculizumab. The prompt disease remission after eculizumab treatment, both at the onset and during recurrences, supports the recent idea that the alternative complement pathway is activated in the presence of ADAMTS13 deficiency and suggests that complement plays a pathogenetic role in microvascular thrombosis (Noris M, et al., Nat. Rev. Nephrol. 2012; 8(11):622-633). In one study of 23 patients with acquired TTP and anti-ADAMTS13 antibodies, plasma levels of complement activation markers (C3a and sC5b-9) were found to be elevated during the acute phase and normalized at remission (see Reti M, et al., J. Thromb. Haemost., 2012; 10(5):791-798). In addition, it was previously documented that sera from 8 patients (4 with acquired TTP and 4 with congenital TTP) cause more C3 and C5b-9 deposits on microvascular endothelial cells than sera from controls (Ruiz-Torres M P, et al., Thromb. Haemost. 2005; 93(3):443-452). More recently, glomerular C3 and C5b-9 deposits have been reported in the kidney biopsy specimens of 2 patients with congenital TTP, thus confirming that in TTP, complement is activated in the renal microvasculature (see Tati R, et al., J. Immunol. 2013; 191(5): 2184-2193. How complement is activated in TTP is a matter of intensive investigation. Recent in vitro studies have documented that components of the alternative complement pathway (C3, CFB, CFD, and properdin) bind endothelial cell-anchored ULvWF chains, which precede the formation of the C3 convertase (see Tati R, et al., J. Immunol. 2013; 191(5):2184-2193; and Turner N A, et al., PLoS One. 2013; 8(3):e59372). These findings, together with the recognized role of ADAMTS13 in cleaving ULvWF into smaller multimers, offer a plausible molecular explanation for complement activation in TTP and for the rapid and dramatic response to anti-C5 treatment we observed in the patient described here. Furthermore, while smaller vWF multimers favor the degradation of the C3 activation product C3b by CFI, ULvWF multimers, which are present in the circulation of patients with TTP, do not (Feng S, et al., Blood. 2015; 125(6):1034-1037), which further supports the hypothesis that ADAMTS13 plays a role in modulating the alternative complement pathway. In this patient, ADAMTS13 deficiency could conceivably have caused excessive assembly and impaired the degradation of complement components on the ULvWF multimers anchored to the microvascular endothelium, as well as complement-mediated injury, mimicking the events associated with genetic complement dysregulation of aHUS. Chapin et al. described a patient given a diagnosis of TTP due to ADAMTS13 deficiency with anti-ADAMTS13 antibodies, which was refractory to plasma exchange, glucocorticoids, rituximab, and vincristine, but eventually responded to eculizumab (see Chapin J, et al., Br. J. Haematol. 2012; 157(6):772-774). However, plasma samples taken during relapses were positive for anti-CFH antibodies, which have been found in 5% to 10% of patients with aHUS, and the authors concluded that this exceptional case had a coexistent disease process involving both TTP and aHUS (see Dragon-Durey M A, et al., J. Am. Soc. Nephrol. 2005; 16(2):555-563; and Tsai E, et al., Br. J. Haematol. 2013; 162(4):558-559). Given that extensive investigation did not reveal a known genetic or acquired complement abnormality in the case reported here and because the patient achieved remission after treatment solely with eculizumab, it is hypothesized that eculizumab may control TMA in the setting of congenital ADAMTS13 deficiency.

SEQUENCE SUMMARY

SEQ ID NO: 1
amino acid sequence of heavy chain CDR1 of
eculizumab (as defined under combined Kabat-
Chothia definition)
GYIFSNYWIQ
SEQ ID NO: 2
amino acid sequence of heavy chain CDR2 of
eculizumab (as defined under Kabat
definition)
EILPGSGSTEYTENFKD
SEQ ID NO: 3
amino acid sequence of the heavy chain CDR3 of
eculizumab (as defined under combined Kabat
definition).
YFFGSSPNWYFDV
SEQ ID NO: 4
amino acid sequence of the light chain CDR1 of
eculizumab (as defined under Kabat definition)
GASENIYGALN
SEQ ID NO: 5
amino acid sequence of light chain CDR2 of
eculizumab (as defined under Kabat definition)
GATNLAD
SEQ ID NO: 6
amino acid sequence of light chain CDR3 of
eculizumab (as defined under Kabat definition)
QNVLNTPLT
SEQ ID NO: 7
amino acid sequence of heavy chain variable region
of eculizumab
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSS
SEQ ID NO: 8
amino acid sequence of light chain variable region
of eculizumab, BNJ441 antibody, and BNJ421
antibody
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK
SEQ ID NO: 9
amino acid sequence of heavy chain constant region
of eculizumab and BNJ421 antibody
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLGK
SEQ ID NO: 10
amino acid sequence of entire heavy chain of
eculizumab
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 11
amino acid sequence of entire light chain of
eculizumab, BNJ441 antibody, and BNJ421 antibody
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 12
amino acid sequence of heavy chain variable region
of BNJ441 antibody and BNJ421 antibody
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS
SEQ ID NO: 13
amino acid sequence of heavy chain constant region
of BNJ441 antibody
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK
SEQ ID NO: 14
amino acid sequence of entire heavy chain of
BNJ441 antibody
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS ASTKGPSVFPLAPCSRSTSESTAALGC
LVKDYFPEPVTVSWNSGALTSGVHTFPAVLQSSGLYSLSSVVTVPSSNFG
TQTYTCNVDHKPSNTKVDKTVERKCCVECPPCPAPPVAGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVL
DSDGSFFLYSRLTVDKSRWQEGNVFSCSV [symbol] HEALH [symbol] HYTQKSLSLSLGK
SEQ ID NO: 15
amino acid sequence of IgG2 heavy chain constant
region variant comprising YTE substitutions
ASTKGPSVFPLAPCSRSTSESTAALGCLVKDYFPEPVTVSWNSGALTSGV
HTFPAVLQSSGLYSLSSVVTVTSSNFGTQTYTCNVDHKPSNTKVDKTVER
KCCVECPPCPAPPVAGPSVFLFPPKPKDTLYITREPEVTCVVVDVSHEDP
EVQFNWYVDGMEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK
SEQ ID NO: 16
amino acid sequence of entire heavy chain of
eculizumab variant comprising heavy chain constant
region depicted in SEQ ID NO: 15 (above)
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVTSSNFGT
QTYTCNVDHKPSNTKVDKTVERKCCVECPPCPAPPVAGPSVFLFPPKPKD
TLYITREPEVTCVVVDVSHEDPEVQFNWYVDGMEVHNAKTKPREEQFNST
FRVVSVLTVVHQDWLNGKEYKCKVSNKGLPAPIEKTISKTKGQPREPQVY
TLPPSREEMTKNQVSLTCLVKGFYPSDIAVEWESNGQPENNYKTTPPMLD
SDGSFFLYSKLTVDKSRWQQGNVFSCSVMHEALHNHYTQKSLSLSPGK
SEQ ID NO: 17
amino acid sequence of light chain CDR1 of
eculizumab (as defined under Kabat definition)
with glycine to histidine substitution at position
8 relative to SEQ ID NO: 4
GASENIYHALN
SEQ ID NO: 18
depicts amino acid sequence of heavy chain CDR2 of
eculizumab in which serine at position 8 relative
to SEQ ID NO: 2 is substituted with histidine
EILPGSGHTEYTENFKD
SEQ ID NO: 19
amino acid sequence of heavy chain CDR1 of
eculizumab in which tyrosine at position 2 (rela-
tive to SEQ ID NO: 1) is substituted with
histidine
GHIFSNYWIQ
SEQ ID NO: 20
amino acid sequence of entire heavy chain of
BNJ421 antibody
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSV [symbol] HEALH [symbol] HYTQKSLSLSLGK

1. A method of treating a human pediatric patient with an ADAMTS13 deficiency, the method comprising administering to the patient an anti-C5 antibody, or antigen binding fragment thereof, comprising CDR1, CDR2, and CDR3 heavy chain sequences as set forth in SEQ ID NOs: 1, 2, and 3, respectively, and CDR1, CDR2, and CDR3 light chain sequences as set forth in SEQ ID NOs: 4, 5, and 6, respectively.

2. A method of treating a human pediatric patient with congenital Thrombotic Thrombocytopenic Purpura, the method comprising administering to the patient an anti-C5 antibody, or antigen binding fragment thereof, comprising CDR1, CDR2, and CDR3 heavy chain sequences as set forth in SEQ ID NOs: 1, 2, and 3, respectively, and CDR1, CDR2, and CDR3 light chain sequences as set forth in SEQ ID NOs: 4, 5, and 6, respectively.

3. The method of claim 1, wherein the anti-C5 antibody, or antigen-binding fragment thereof, comprises a heavy chain variable region as set forth in SEQ ID NO: 7 and a light chain variable region as set forth in SEQ ID NO: 8.

4. The method of claim 1, wherein the anti-C5 antibody, or antigen-binding fragment thereof, comprises a heavy chain comprising the amino acid sequence depicted in SEQ ID NO: 9 and a light chain comprising the amino acid sequence depicted in SEQ ID NO: 10.

5. The method of claim 1, wherein the anti-C5 antibody is eculizumab.

6. The method of claim 1, wherein the anti-C5 antibody, or antigen binding fragment thereof, is administered (a) weekly at a dose of 900 mg for four weeks and (b) once every two weeks thereafter at a dose of 1,200 mg.

7. The method of claim 1, wherein the anti-C5 antibody, or antigen binding fragment thereof, is administered intravenously.

8. The method of claim 1, wherein the anti-C5 antibody, or antigen binding fragment thereof, is administered for at least 20, 30, or 40 weeks.

9. The method of claim 1, wherein the human pediatric patient has ultra large von Willebrand factor (ULvWF) multimers circulating in the blood prior to treatment with an anti-C5 antibody, or antigen binding fragment thereof.

10. The method of claim 1, wherein the human pediatric patient has elevated levels of C3a and/or sC5b-9 in the plasma prior to treatment with an anti-C5 antibody, or antigen binding fragment thereof.

11. (canceled)

12. The method of claim 1, wherein the human pediatric patient has elevated levels of C3 glomerular deposits in kidney biopsy specimens and/or has elevated levels of serum-induced C5b-9 deposits on endothelial cells ex-vivo, prior to treatment with an anti-C5 antibody, or antigen binding fragment thereof.

13. (canceled)

14. The method of claim 1, wherein the ADAMTS13 deficiency is associated with one or more ADAMTS13 gene mutations.

15. The method of claim 14, wherein the ADAMTS13 mutation is a guanine to adenine change at nucleotide 3,251.

16. The method of claim 14, wherein the ADAMTS13 mutation is a deletion of a cytosine at nucleotide 4,049.

17. The method of claim 14, wherein the ADAMTS13 deficiency is associated with two ADAMTS13 mutations, wherein the first ADAMTS13 mutation is a guanine to adenine change at nucleotide 3,251 and the second ADAMTS13 mutation is a deletion of a cytosine at nucleotide 4,049.

18. The method of claim 1, wherein the ADAMTS13 deficiency is determined by undetectable levels of ADAMTS13 activity, as assessed by a collagen-binding assay and/or fluorescence resonance energy transfer (FRET).

19. The method of claim 1, further comprising administering an antimeningococcal vaccine and/or antibiotics prior to administering the anti-C5 antibody, or antigen binding fragment thereof.

20. The method of claim 1, wherein the treatment results in a normalized platelet count, normalized lactate dehydrogenase (LDH) and serum creatinine levels, and normalized diuresis.

21. (canceled)

22. (canceled)

23. A kit for treating a human pediatric patient with an ADAMTS13 deficiency, the kit comprising:
(a) a dose of an anti-C5 antibody, or antigen binding fragment thereof, comprising CDR1, CDR2 and CDR3 domains of the heavy chain variable region having the sequence set forth in SEQ ID NO: 7, and CDR1, CDR2 and CDR3 domains of the light chain variable region having the sequence set forth in SEQ ID NO: 8; and
(b) instructions for using the anti-C5 antibody, or antigen binding fragment thereof, in the method of claim 1.

24. A kit for treating a human pediatric patient with congenital Thrombotic Thrombocytopenic Purpura, the kit comprising:
(a) a dose of an anti-C5 antibody, or antigen binding fragment thereof, comprising CDR1, CDR2 and CDR3 domains of the heavy chain variable region having the sequence set forth in SEQ ID NO: 7, and CDR1, CDR2 and CDR3 domains of the light chain variable region having the sequence set forth in SEQ ID NO: 8; and
(b) instructions for using the anti-C5 antibody, or antigen binding fragment thereof, in the method of claim 2.

25. A method of treating a human pediatric patient with an ADAMTS13 deficiency, the method comprising intravenously administering eculizumab to the patient (a) weekly at a dose of 900 mg for four weeks and (b) once every two weeks thereafter at a dose of 1,200 mg.

26. A method of treating a human pediatric patient with congenital Thrombotic Thrombocytopenic Purpura, the method comprising intravenously administering eculizumab to the patient (a) weekly at a dose of 900 mg for four weeks and (b) once every two weeks thereafter at a dose of 1,200 mg.

27. The method of claim 2, wherein the anti-C5 antibody, or antigen-binding fragment thereof, comprises a heavy chain variable region as set forth in SEQ ID NO: 7 and a light chain variable region as set forth in SEQ ID NO: 8.

28. The method of claim 2, wherein the anti-C5 antibody, or antigen-binding fragment thereof, comprises a heavy chain comprising the amino acid sequence depicted in SEQ ID NO: 9 and a light chain comprising the amino acid sequence depicted in SEQ ID NO: 10.

29. The method of claim 2, wherein the anti-C5 antibody is eculizumab.

30. The method of claim 2, wherein the anti-C5 antibody, or antigen binding fragment thereof, is administered (a) weekly at a dose of 900 mg for four weeks and (b) once every two weeks thereafter at a dose of 1,200 mg.

31. The method of claim 2, wherein the anti-C5 antibody, or antigen binding fragment thereof, is administered intravenously.

32. The method of claim 2, wherein the anti-C5 antibody, or antigen binding fragment thereof, is administered for at least 20, 30, or 40 weeks.

33. The method of claim 2, wherein the human pediatric patient has ultra large von Willebrand factor (ULvWF) multimers circulating in the blood prior to treatment with an anti-C5 antibody, or antigen binding fragment thereof.

34. The method of claim 2, wherein the human pediatric patient has elevated levels of C3a and/or sC5b-9 in the plasma prior to treatment with an anti-C5 antibody, or antigen binding fragment thereof.

35. The method of claim 2, wherein the human pediatric patient has elevated levels of C3 glomerular deposits in kidney biopsy specimens and/or has elevated levels of serum-induced C5b-9 deposits on endothelial cells ex-vivo, prior to treatment with an anti-C5 antibody, or antigen binding fragment thereof.

36. The method of claim 2, further comprising administering an antimeningococcal vaccine and/or antibiotics prior to administering the anti-C5 antibody, or antigen binding fragment thereof.

37. The method of claim 2, wherein the treatment results in a normalized platelet count, normalized lactate dehydrogenase (LDH) and serum creatinine levels, and normalized diuresis.
